(19) United States
(12) Patent Application Publication
(10) Pub. No.: US 2010/0130913 A1
(43) Pub. Date: May 27, 2010

(54) INTEGRATED CATHETER AND PULSE GENERATOR SYSTEMS AND METHODS

(76) Inventors: Tamara Colette Baynham, Piscataway, NJ (US); Steven D. Girouard, Chagrin Falls, OH (US)

Correspondence Address: SCHWEGMAN, LUNDBERG & WOESSNER/BSC-CRM, PO BOX 2938, MINNEAPOLIS, MN, 55402, US

(21) Appl. No.: 12/694,328

(22) Filed: Jan. 27, 2010

Related U.S. Application Data
(63) Continuation of application No. 11/468,875, filed on Aug. 31, 2006.

(51) Int. Cl. A61F 2/84; A61N 1/362; A61M 29/02; A61M 25/10

(52) U.S. Cl. 604/21; 607/3; 606/194; 606/108

(57) ABSTRACT

Disclosed herein, among other things, is a system for providing pacing during revascularization. An embodiment of the system includes an angioplasty or stent delivery catheter system having a catheter, a balloon and an inflation device adapted to inflate and deflate the balloon for delivery of a stent. The embodiment also includes a programmable pulse generator and at least one electrode integrated with the angioplasty catheter system, where the pulse generator is connected to the electrode. In various embodiments, at least one integrated sensor is connected to the angioplasty catheter system. The sensor is adapted to sense a parameter indicative of flow restoration and trigger the pulse generator to begin pacing based on the parameter.

### CROSS REFERENCE TO RELATED APPLICATIONS

This application is a continuation of U.S. application Ser. No. 11/468,875, filed Aug. 31, 2006, which is hereby incorporated by reference in its entirety.

The following commonly assigned U.S. patent application is related to the present application and is incorporated herein by reference in its entirety: “Method and Apparatus for Pacing During Revascularization,” Ser. No. 11/113,828, filed on Apr. 25, 2005.

### TECHNICAL FIELD

This disclosure relates generally to medical devices, and more particularly integrated catheter and pulse generator systems and methods.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the body organs and pump it to the lungs where the blood gets oxygenated. Contractions of the myocardium (cardiac muscles) produce these pumping functions. In a normal heart, the sinoatrial node, the heart's natural pacemaker, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract in synchrony to result in efficient pumping functions. A blocked or otherwise abnormal electrical conduction system and/or deteriorated myocardial tissue cause dysynchronous contraction of the heart, resulting in poor hemodynamic performance, including a diminished blood supply to the heart and the rest of the body. The condition where the heart fails to pump enough blood to meet the body's metabolic demand is known as heart failure.

Myocardial infarction (MI) is the necrosis of portions of the myocardial tissue resulted from cardiac ischemia, a condition in which the myocardium is deprived of adequate oxygen and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is diminished, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance, a significantly increased risk of developing heart failure and an increased risk of sudden cardiac death.

When a blood vessel such as the coronary artery is partially or completely occluded, a revascularization procedure such as percutaneous transluminal coronary angioplasty (PCTA) can be performed to reopen the occluded blood vessel. Revascularization is also commonly accomplished by combining the PCTA procedure with the delivery of a coronary stent to the affected region to maintain patency of the artery. The act of revascularization may result in additional injury to the cardiac tissue, termed reperfusion injury. Upon resumption of flow (reperfusion) several events are triggered such as an increase in oxygen free radicals, altered calcium ion (Ca²⁺) handling, altered metabolism, microvascular endothelial dysfunction, and platelet and neutrophil activation leading to reperfusion injury. Reperfusion injury may lead to stunned myocardium, no reflow phenomenon, and lethal reperfusion with myocyte necrosis. In addition, the revascularization procedure itself involves a temporary occlusion of the coronary artery. In addition, plaques dislodged and displaced by the revascularization procedure may enter small blood vessels branching from the blood vessel in which the revascularization is performed, causing occlusion of these small blood vessels. The plaque dislodged during the revascularization procedure may also cause distal embolization. The temporary occlusion, or displacement and dislodgement of plaque, may cause cardiac injuries such as further expansion of the region of infarcted tissue. In addition, the revascularization procedure is known to increase the risk for occurrences of arrhythmia.

Providing pacing during revascularization can reduce the damage caused by reperfusion injury as well as the probability of arrhythmia during the revascularization process. Improved systems and methods for providing this therapy are needed.

### SUMMARY

The above-mentioned problems and others not expressly discussed herein are addressed by the present subject matter and will be understood by reading and studying this specification.

Disclosed herein, among other things, is an angioplasty or stent delivery catheter system. According to one embodiment, the angioplasty catheter system includes a catheter, a balloon and an inflation device adapted to inflate and deflate the balloon for delivery of a stent. The embodiment also includes a programmable pulse generator and at least one electrode integrated with the angioplasty catheter system, where the pulse generator is connected to the electrode. The pulse generator is programmably controlled by an external device via a radio frequency (RF) link, according to varying embodiments. According to an embodiment, the balloon has a channel or lumen embedded that allows for flow during inflation that would provide the ability to deliver cells or other therapeutics.

Disclosed herein, among other things, is a catheter system capable of delivering a self-expanding stent to an occluded artery. According to one embodiment, the catheter system includes a catheter, a self expanding stent and a mechanical device for releasing the self expanding stent in a desired anatomic location. The embodiment also includes a programmable pulse generator and at least one electrode integrated with the self-expanding stent catheter system, where the pulse generator is connected to the electrode. The pulse generator is programmably controlled by an external device via wireless communication, according to varying embodiments.

Another embodiment includes an angioplasty catheter system, where the angioplasty catheter system includes a catheter, a balloon and an inflation device adapted to inflate and deflate the balloon. The embodiment also includes a programmable pulse generator and at least one electrode integrated with the angioplasty catheter system, where the pulse generator is connected to the electrode. The embodiment further includes at least one integrated sensor connected to the angioplasty catheter system. The sensor is adapted to sense a parameter indicative of flow restoration and trigger the pulse generator to begin pacing based on the parameter, according to various embodiments.

Disclosed herein, among other things, is a method for applying electrical therapy. According to an embodiment, the method includes performing angioplasty therapy using a catheter-based system, where the system includes a catheter, a balloon and an inflation device adapted to inflate and deflate the balloon. The embodiment also includes providing cardioprotective pacing during the therapy using a programmable pulse generator integrated with the catheter-based system. In various embodiments, the method further includes sensing at least one parameter indicative of flow restoration.

Disclosed herein, among other things, is a method for applying cell therapy. According to an embodiment, the method includes delivering cells into areas of myocardial infarction using an angioplasty catheter system having a programmable pulse generator integrated with the system. The embodiment also includes providing pacing from the pulse generator to improve integration or differentiation of the cells.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

FIG. 1 illustrates a block diagram of an angioplasty or stent delivery catheter system, according to one embodiment.

FIGS. 2A-2C illustrate block diagrams of angioplasty or stent delivery catheter systems, according to various embodiments.

FIGS. 3A-3B illustrate block diagrams of angioplasty or stent delivery catheter systems including sensor(s), according to various embodiments.

FIG. 4 illustrates a block diagram of a system with a pulse generator, according to one embodiment.

FIG. 5 illustrates a block diagram of a programmer such as illustrated in the system of FIG. 4 or other external device to communicate with the pulse generator(s), according to one embodiment.

FIG. 6 illustrates a flow diagram of a method for applying electrical therapy, according to one embodiment.

FIG. 7 illustrates a flow diagram of a method for applying cell therapy, according to one embodiment.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

Various embodiments of the present subject matter are related to angioplasty or stent delivery catheter systems. In various embodiments, the present subject matter includes one or more pulse generators integrated with an angioplasty catheter system. In various embodiments, these angioplasty catheter systems with integrated pulse generators are used to provide cardioprotective pacing therapy during revascularization. The described pacing system minimizes damage to the myocardial tissue and preventing arrhythmias during a revascularization procedure that requires temporary occlusion of one or more blood vessels. In a specific application, this system provides for cardiac protection pacing during a percutaneous transluminal coronary angioplasty (PTCA) procedure. Cardiac protection pacing includes the delivery of a pacing therapy before, during, and/or after the temporary occlusion of a coronary artery associated with the PTCA procedure, for preventing or reducing adverse effects of the occlusion, which is an ischemic event. The pacing therapy can be delivered at almost any time during a revascularization procedure, as soon as pacing electrodes are in place, without substantially interfering with the revascularization procedure. In one embodiment, the cardiac protection pacing sequence includes alternating pacing and non-pacing periods. Each pacing period is a pacing duration during which the pacing pulses are delivered in a predetermined pacing mode. The non-pacing period is a non-pacing duration during which no pacing pulses is delivered. In one embodiment, during each pacing period, rapid, asynchronous pacing is applied. In other words, pacing pulses are delivered at a rate substantially higher than the patient's intrinsic heart rate without being synchronized to the patient's intrinsic cardiac contractions. One embodiment of a cardiac protection pacing sequence includes two cycles of alternating pacing and non-pacing periods. In one embodiment, the number of the cycles of alternating pacing and non-pacing periods is programmable, and each of the pacing and non-pacing periods is programmable. In some embodiments, the angioplasty catheter systems with integrated pulse generators are used to improve cell integration and differentiation during cell therapy, such as stem cell therapy used to restore function after a myocardial infarction (MI). In other embodiments, the angioplasty catheter systems with integrated pulse generators are used to stimulate electrically-active promoters used to locally control gene expression.

As defined herein, having a pulse generator “integrated with” an angioplasty or stent delivery catheter system includes having the pulse generator sized and positioned within the catheter system, so that the pulse generator is inserted into and removed from a human body with the catheter system. In various embodiments, this involves having a pulse generator with smaller dimensions than conventional implantable pulse generators that are chronically implanted (such as pacemakers and defibrillators).

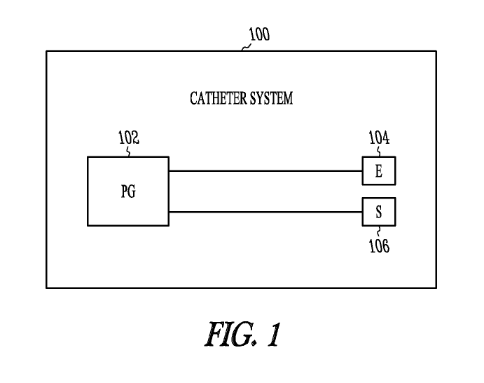

FIG. 1 illustrates a block diagram of an angioplasty (or stent delivery) catheter system, according to one embodiment. The embodiment includes an angioplasty catheter system 100 and a programmable pulse generator 102 integrated with the angioplasty catheter system. According to various embodiments, the angioplasty catheter system 100 further includes at least one electrode 104, and the pulse generator 102 is connected to the at least one electrode. The angioplasty catheter system 100 further includes at least one sensor 106, and the pulse generator 102 is connected to the at least one sensor, according to various embodiments.

The electrode, or plurality of electrodes, is embedded in a distal catheter body, in an embodiment. The electrodes may be placed in a number of positions in the angioplasty catheter system, according to varying embodiments. Additional information on electrode placement can by found in application Ser. No. 11/113,828, that has previously been incorporated by reference.

According to various embodiments, pulse generators 102 include devices that function as various cardiac rhythm management (CRM) devices such as pacemakers, cardioverters, defibrillators, cardiac resynchronization therapy (CRT) devices, as well as combination devices that provide more than one of these therapy modalities to a subject. The pulse generator is programmably controlled by an external device via wireless communication, according to various embodiments. Examples of types of wireless communication used include, but are not limited to, radio frequency (RF) links and inductive telemetry. Examples of external devices include, but are not limited to, programmers (such as depicted in FIG. 5) and remote patient monitoring systems. A pacing algorithm starts automatically (such as upon deflation of a balloon in the catheter system) or when an operator activates the pulse generator. The RF link is used to download pacing routines, parameters for the routines, or to switch between predefined routines, in an embodiment. The pulse generator is powered by an internal or external battery, or a combination of internal and external batteries, in varying embodiments. In one embodiment, the pulse generator is adapted to be charged by the external battery prior to use. In various embodiments, the pulse generator has a pacing output in the range from sub-threshold to high-output (5 to 20 times the threshold) pacing. High-output pacing is used to target neurotransmitters, in varying embodiments. Pacing includes anodal pacing or multi-site pacing (using a catheter or guide wire with multiple active poles), or both, in various embodiments. Various embodiments of the pacing electrodes have unipolar or multi-polar configurations. Unipolar configurations use an external patch or return electrode along the length of the catheter, in various embodiments.

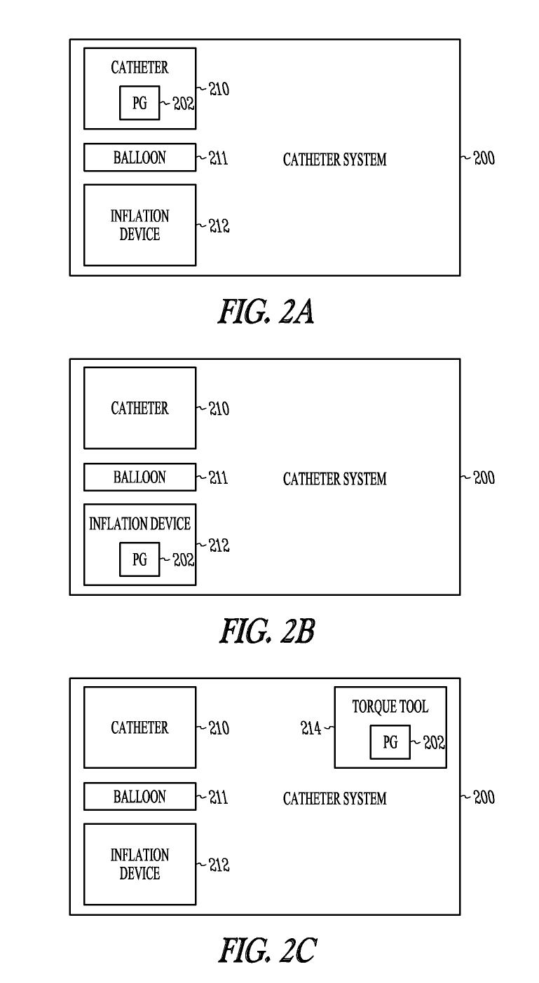

FIGS. 2A-2C illustrate block diagrams of angioplasty or stent delivery catheter systems, according to various embodiments. In FIG. 2A, the angioplasty catheter system 200 includes a catheter 210, a balloon 211, and an inflation device 212 adapted to inflate and deflate the balloon for delivery of a stent, and the pulse generator 202 is integrated with the catheter 210. In FIG. 2B, the angioplasty catheter system 200 includes a catheter 210, a balloon 211, and an inflation device 212 adapted to inflate and deflate the balloon, and the pulse generator 202 is integrated with the inflation device 212. In FIG. 2C, the angioplasty catheter system 200 includes a catheter 210, a balloon 211, an inflation device 212, and a torquing tool 214, and the pulse generator 202 is integrated with the torquing tool. According to various embodiments, the pulse generator is sized to fit within the angioplasty catheter system, and is placed in a number of locations within the system, including but not limited to those locations depicted in FIGS. 2A-2C.

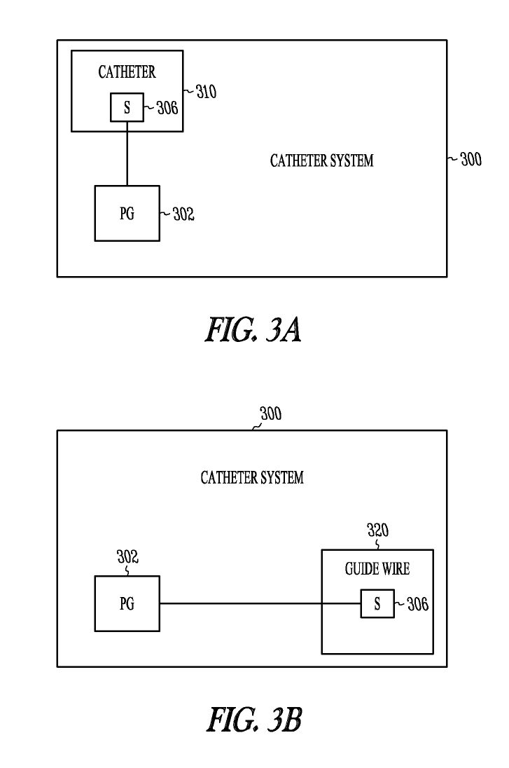

FIGS. 3A-3B illustrate block diagrams of angioplasty or stent delivery catheter systems including sensor(s), according to various embodiments. An embodiment includes an angioplasty catheter system 300 and a programmable pulse generator 302 integrated with the angioplasty catheter system. The embodiment further includes at least one integrated sensor 306 connected to the angioplasty catheter system. The sensor is adapted to sense a parameter indicative of flow restoration and trigger the pulse generator to begin pacing based on the parameter, according to various embodiments. In FIG. 3A, the sensor 306 is integrated with the catheter 310. In FIG. 3B, the sensor 306 is integrated with a guide wire 320 or guide catheter. According to an embodiment, the guide wire is adapted to function as a pacing lead. The sensor is sized to fit within the angioplasty catheter system, and is placed in a number of locations within the system, including but not limited to those locations depicted in FIGS. 3A-3B. Multiple sensors are used in multiple locations, in various embodiments. The sensors are used as part of a closed-loop system, and sensor outputs drive the initiation of and parameters for the post-conditioning pacing routine, in varying embodiments.

According to various embodiments, the sensor includes a flow sensor, a temperature sensor, an accelerometer, or a chemical sensor such as an oxygen (pO₂) sensor, a carbon dioxide (pCO₂) sensor, or a hydrogen (pH) sensor. Other types of sensors may be used without departing from the scope of this disclosure. According to varying embodiments, the catheter system includes the balloon portion with a channel (or lumen) embedded that allows for flow during inflation that would provide the ability to deliver cells and/or other therapeutics. In other embodiments, the lumen is embedded in the catheter.

Disclosed herein, among other things, is a catheter system capable of delivering a self-expanding stent to an occluded artery. Types of self-expanding stents include, but are not limited to, nitinol stents. These systems have a catheter that rides over a wire to deliver the stent, but there is no balloon to expand the stent. A mechanical system dislodges the stent into the correct position and the stent self expands in place to open the artery. According to one embodiment, the catheter system includes a catheter, a self expanding stent and a mechanical device for releasing the self expanding stent in a desired anatomic location. The embodiment also includes a programmable pulse generator and at least one electrode integrated with the self-expanding stent catheter system, where the pulse generator is connected to the electrode. The pulse generator is programmably controlled by an external device via wireless communication, according to varying embodiments. The system further includes a guide wire, and the guide wire is adapted to function as a pacing lead, according to various embodiments.

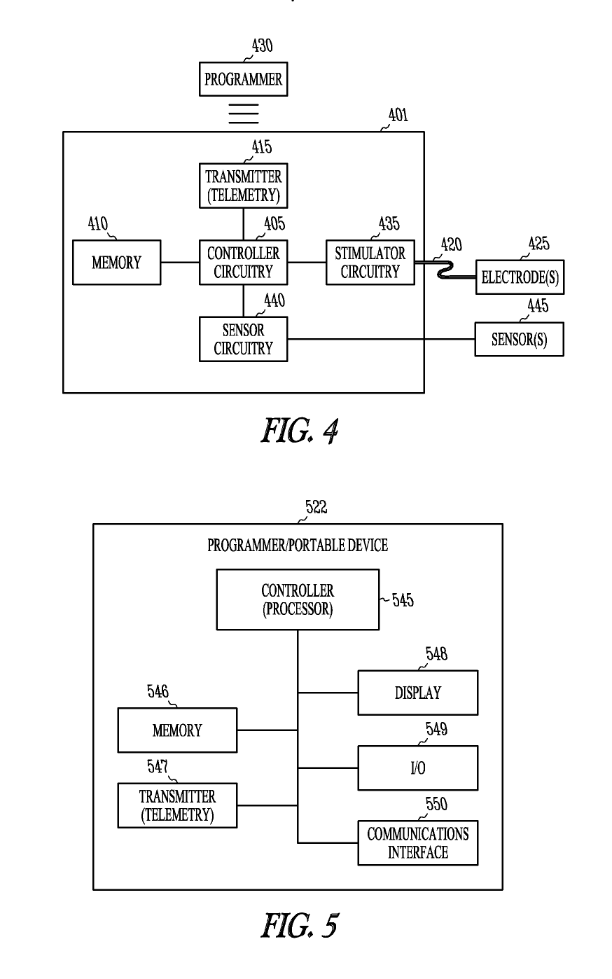

FIG. 4 illustrates a block diagram of a system with a pulse generator such as the pulse generator illustrated in the system of FIG. 1, according to one embodiment. The system includes a pulse generator 401, an electrical lead 420 coupled to the pulse generator 401, and at least one electrode 425. The pulse generator includes a controller circuit 405, a memory circuit 410, a telemetry circuit 415, and a stimulation circuit 435. The controller circuit 405 is operable on instructions stored in the memory circuit to deliver an electrical stimulation therapy. Therapy is delivered by the stimulation circuit 435 through the lead 420 and the electrode(s) 425. The telemetry circuit 415 allows communication with an external programmer 430. The programmer 430 is used to adjust the programmed therapy provided by the pulse generator 401, and the pulse generator reports device data (such as battery capacity and lead resistance) and therapy data (such as sense and stimulation data) to the programmer using radio telemetry, for example. The illustrated system also includes sensor circuitry 440 that is connected to at least one integrated sensor 445 connected to an angioplasty catheter system. According to various embodiments, the sensor 445 is adapted to sense a parameter indicative of flow restoration and trigger the pulse generator to begin pacing based on the parameter. According to various embodiments, the disclosed systems and methods are used with a leadless device. For example, in an embodiment, one or more satellite electrodes are controlled wirelessly to deliver electrical therapy.

FIG. 5 illustrates a block diagram of a programmer such as illustrated in the system of FIG. 4 or other external device to communicate with the pulse generator(s), according to one embodiment. FIG. 5 illustrates a programmer 522, such as the programmer 430 illustrated in the system of FIG. 4 or other external device to communicate with the medical device(s), according to one embodiment. Examples of other external devices include Personal Digital Assistants (PDAs), personal laptop and desktop computers in a remote patient monitoring system, or a handheld device in such a system. The illustrated device 522 includes controller circuitry 545 and a memory 546. The controller circuitry 545 is capable of being implemented using hardware, software, and combinations of hardware and software. For example, according to various embodiments, the controller circuitry 545 includes a processor to perform instructions embedded in the memory 546 to perform a number of functions, including communicating data and/or programming instructions to the devices. The illustrated device 522 further includes a transceiver 547 and associated circuitry for use to communicate with a device. Various embodiments have wireless communication capabilities. For example, various embodiments of the transceiver 547 and associated circuitry include a telemetry coil for use to wirelessly communicate with a device. The illustrated device 522 further includes a display 548, input/output (I/O) devices 549 such as a keyboard or mouse/pointer, and a communications interface 550 for use to communicate with other devices, such as over a communication network.

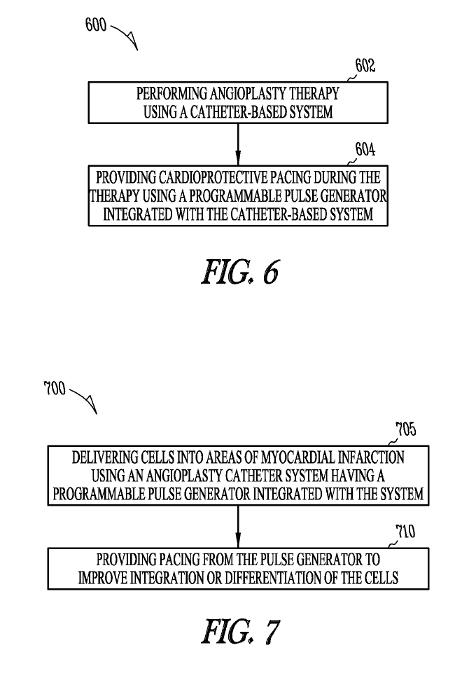

FIG. 6 illustrates a flow diagram of a method for applying electrical therapy, according to one embodiment. According to an embodiment, the method 600 includes performing angioplasty therapy using a catheter-based system, at 602. The method embodiment also includes providing cardioprotective pacing during the therapy using a programmable pulse generator integrated with the catheter-based system, at 604. In various embodiments, the method further includes sensing at least one parameter indicative of flow restoration. The method includes triggering the pulse generator to begin pacing based on the parameter, according to varying embodiments. In one embodiment, providing cardioprotective pacing includes providing pacing to stimulate electrically-active promoters used to locally control gene expression. In another embodiment, providing cardioprotective pacing includes triggering the pulse generator to run a predefined script. Providing cardioprotective pacing includes triggering an alarm to allow a physician to control therapy, in various embodiments. The method is beneficial for use in a variety of patients, including acute MI, refractory angina and post-MI patients. The method is convenient, easy to use, and is an effective solution for these patients.

FIG. 7 illustrates a flow diagram of a method for applying cell therapy, according to one embodiment. According to an embodiment, the method 700 includes delivering cells into areas of myocardial infarction using an angioplasty catheter system having a programmable pulse generator integrated with the system, at 705. The method embodiment also includes providing pacing from the pulse generator to improve integration or differentiation of the cells, at 710. According to one embodiment, providing pacing includes providing pacing to improve integration of cells into areas of myocardial infarction. According to another embodiment, providing pacing includes providing pacing to improve differentiation of cells into areas of myocardial infarction. According to further embodiment, providing pacing includes providing pacing to improve integration and differentiation of cells into areas of myocardial infarction. Types of cells used in this therapy include, but are not limited to, stem cells and biological tissue cells. Types of stem cells used in this therapy include, for example, adult stem cells, bone-marrow derived stem cells, and embryonic stem cells.

Although specific embodiments have been illustrated and described herein, it will be appreciated by those of ordinary skill in the art that any arrangement which is calculated to achieve the same purpose may be substituted for the specific embodiment shown. This application is intended to cover adaptations or variations of the present subject matter. It is to be understood that the above description is intended to be illustrative, and not restrictive. Combinations of the above embodiments, and other embodiments will be apparent to those of skill in the art upon reviewing the above description. The scope of the present subject matter should be determined with reference to the appended claims, along with the full scope of equivalents to which such claims are entitled.

We claim:

1. A system for delivering therapy to a human body, comprising:
an angioplasty catheter system, wherein the angioplasty catheter system includes a catheter, a balloon and an inflation device adapted to inflate and deflate the balloon for delivery of a stent;
a programmable pulse generator integrated with the catheter system to be inserted and removed from the human body with the catheter system, and programmed to provide cardioprotective pacing therapy during revascularization, and wherein the cardioprotective pacing includes a programmed sequence of pacing where the pacing pulses are delivered at a higher rate than the patient's intrinsic heart rate; and
at least one electrode integrated with the angioplasty catheter system, wherein the pulse generator is connected to the electrode.

2. The system of claim 1, wherein the pulse generator includes a pacemaker.

3. The system of claim 1, wherein the pulse generator is programmably controlled by an external device via wireless communication.

4. The system of claim 3, wherein the external device includes a programmer.

5. The system of claim 3, wherein the external device includes a remote patient monitoring system.

6. The system of claim 1, wherein the pulse generator is powered by an external battery.

7. The system of claim 6, wherein the pulse generator is adapted to be charged by the external battery prior to use.

8. The system of claim 1, further comprising:
at least one integrated sensor connected to the angioplasty catheter system, the sensor adapted to sense a parameter indicative of flow restoration and trigger the pulse generator to begin pacing based on the parameter.

9. The system of claim 8, wherein the sensor includes a flow sensor.

10. The system of claim 8, wherein the sensor includes a temperature sensor.

11. The system of claim 8, wherein the sensor includes an accelerometer.

12. The system of claim 8, wherein the sensor includes a chemical sensor.

13. The system of claim 12, wherein the chemical sensor includes an oxygen (pO₂) sensor.

14. The system of claim 12, wherein the sensor includes a carbon dioxide (pCO₂) sensor.

15. The system of claim 12, wherein the sensor includes a hydrogen (pH) sensor.

16. The system of claim 8, wherein the sensor is integrated with the catheter.

17. The system of claim 8, further comprising a guide wire, and wherein the sensor is integrated with the guide wire.

18. The system of claim 17, wherein the guide wire is adapted to function as a pacing lead.

19. The system of claim 8, wherein the catheter system includes a lumen within the balloon.

20. The system of claim 19, wherein the lumen is adapted to deliver cells.

21. The system of claim 8, wherein the catheter is part of a stent delivery system.

22. A method of applying therapy to a patient, comprising:
performing angioplasty therapy using a catheter-based system, wherein the system includes a catheter, a balloon and an inflation device adapted to inflate and deflate the balloon; and
providing cardioprotective pacing during revascularization using a programmable pulse generator integrated with the catheter system, wherein the pulse generator is configured to be inserted and removed from the patient with the catheter system, and
wherein providing cardioprotective pacing includes providing a programmed sequence of pacing where the pacing pulses are delivered at a higher rate than the patient's intrinsic heart rate.

23. The method of claim 22, further comprising:
sensing at least one parameter indicative of flow restoration.

24. The method of claim 22, wherein providing cardioprotective pacing includes providing pacing to stimulate electrically-active promoters used to locally control gene expression.

25. The method of claim 22, wherein providing cardioprotective pacing includes triggering the pulse generator to run a predefined script.

26. The method of claim 22, wherein providing cardioprotective pacing includes triggering an alarm to allow a physician to control therapy.

27. The system of claim 1, wherein the pulse generator is within the catheter.

28. The system of claim 1, wherein the pulse generator is within the inflation device.

29. The method of claim 22, wherein the patient has an intrinsic cardiac contraction, and wherein providing the programmed sequence of pacing includes providing at least two cycles of alternating pacing and non-pacing periods, and pacing without synchronization to the patient's intrinsic cardiac contraction during each pacing period.
